(19) United States
(12) Patent Application Publication
(10) Pub. No.: US 2020/0207820 A1
(43) Pub. Date: Jul. 2, 2020

(54) ANTIVIRAL, ANTIFIBROTIC AND ANTICANCER ACTIVITIES OF ROYAL-JELLY PROTEINS

(71) Applicants: Salem EL-FIKY, Alexandria (EG); Marwa ABU-SARIE, Alexandria (EG); Noha HABASHY, Alexandria (EG)

(72) Inventors: Salem EL-FIKY, Alexandria (EG); Marwa ABU-SARIE, Alexandria (EG); Noha HABASHY, Alexandria (EG)

(21) Appl. No.: 16/622,506

(22) PCT Filed: Aug. 7, 2017
(86) PCT No.: PCT/EG2017/000022

§ 371 Date: Dec. 19, 2019

(30) Foreign Application Priority Data
Jul. 19, 2017 (EG) 20170701196

(51) Int. Cl. C07K 14/435

(52) U.S. Cl. CPC C07K 14/43572; A61K 38/00

(57) ABSTRACT

This invention discloses purified proteins from Apis mellifera royal jelly (RJ) named major RJ protein 2 and its isoform X1 have proven potent efficacy against HCV and HBV and their complications in the liver “fibrosis and cancer”. Methods for the effective RJ proteins purification, identification, safety and examination against HCV, HBV, fibrosis, and HepG-2 cell line are disclosed. The comparisons with the current most potent anti-HCV drug “Sovaldi” are also disclosed.

### TECHNICAL FIELD

The present invention relates to novel natural compounds having highly potent preventing effects for HCV, HBV and their related diseases. In particular, the present invention relates to two proteins purified from Apis mellifera royal jelly (RJ) named as major royal jelly protein 2 and its isoform X1 having potent anti-HCV and HBV effects, highly improve the liver fibrosis and are moderately cytotoxic against HepG-2 cells.

### BACKGROUND ART

Hepatitis C virus (HCV) and hepatitis B virus (HBV) are the most common etiologies for the liver diseases in the world. Since they are the main causes of liver fibrosis, cirrhosis and hepatocellular carcinoma (HCC). Globally, the prevalence rate of HCV and HBV is approximately 170 and 350 million people worldwide, respectively. More than one million patients die every year from the complications of these viruses, mainly HCC which globally extends in many countries, particularly Asia and Africa. There is no vaccine for HCV infection till now while; an effective vaccine was developed for HBV since 1976.

HCV consists of a single-stranded (+) RNA and is classified into the genus Hepacivirus of the family Flaviviridae. However, HBV is double stranded DNA virus from the genus Orthohepadnavirus of family Hepadnaviridae. Recently, HCV and HBV replication can be observed in vitro using human hepatocellular cancer cell line (HepG-2) and peripheral blood mononuclear cells (PBMCs), which facilitate the evaluation of the antiviral compounds. HCV and HBV can replicate in these cells and therefore these systems are used for identifying the compounds that inhibit the replication of these viruses.

The treatment target for HCV and HBV is to inhibit their replication, reduce inflammation in the liver, reverse fibrosis and stop the progression of cirrhosis and HCC. HCV is treated since 2002 with the interferon-free direct acting antivirals (IFN-free DAA) which are effective drugs able to cure HCV after 8-12 weeks. Sofosbuvir (Sovaldi, SOV) is one of the newest types of these drugs that used with other combinations for the treatment of HCV infection. Despite the efficiency of these drugs, they have many side effects such as osteoporosis, renal toxicity, and pulmonary hypertension. In addition, the intake of these drugs is not safe for pregnant and breast feeding women due to the association of some of them with teratogenic effects in animals. Therefore, none of these DAA has yet been approved by the U.S. Food and Drug Administration (FDA) during pregnancy and lactation. Furthermore, most of these drugs make reactivation for HBV in some HCV infected patients. On the other hand, the treatment strategies for HBV are using IFN/pegylated IFN or nucleoside analogs or their combination for long-term. But, the long-term intake of these drugs is not safe on patients and cause potential renal toxicity besides the possible development of drug resistance. Therefore, the discovery of an effective and safe therapy for HCV and HBV is of great importance and the need of the day.

RJ (bee's milk) is a creamy, whitish product secreted from mandibular and hypopharyngeal glands of nurse bees (Apis mellifera). It is the specific food for the queen bees and helps in their development from the worker bee larva with the age between 5-15 days. RJ is highly acidic substance composed mainly of water (60-70%), proteins (9-18%), sugars (7-18%), and lipids (3-8%) with other compounds. About 80% of the RJ proteins are water soluble belonging to the major RJ protein (MRJP) family, which comprises 9 members (MRJP1-MRJP9) with a molecular mass of 49-87 kDa. To date, the antiviral, antifibrotic and the anticancer activities of RJ and its components are undefined.

### SUMMARY OF THE INVENTION

RJ (FIG. 1) was fractionated into lipid, protein and carbohydrate fractions. The lyophilized fractions of RJ were tested against HCV replication using published methodology. The protein fraction has proven its ex vivo efficacy against HCV thereof, it was further fractionated using a different degree of saturation of ammonium sulfate crystals. Three fractions were tested for their anti-HCV activity and two fractions of them exhibit the potency. The higher protein content fraction from the two potent anti-HCV fractions was further purified using ion exchange chromatography yielding two purified proteins named major RJ protein 2 (MRJP2) and MRJP2 isoform X1.

The two purified MRJPs were identified using matrix assisted laser desorption ionization-time of flight mass spectrometry (MALDI-TOF Ms). The novel MRJP2 isoform X1 is identified as a predicted protein in the database and thus this invention has the priority to confirm its expression from the Apis mellifera genome

The two purified MRJPs have proven their efficacy against HCV and HBV in PBMCs and HepG-2 cells as well as in CCl4-induced fibrosis in hepatocytes and have moderate, not potent anticancer effect. In addition, the two proteins prevent the HCV and HBV cellular entry. The exact anti-HCV and anti-HBV mechanism of the RJ proteins is under investigation.

The acute toxicity of the RJ on the Albino rats was tested. RI has proven its safety on the animal vital organs and interestingly multiple activities were observed like hypolipidemia and enhancing kidney function.

All the studied activities for the purified RJ proteins were compared with Sovaldi. Although Sovaldi showed more potent anticancer effect than the RJ proteins, it exhibited less anti-HCV and less antifibrotic activities. Not only this but also SOV increases the activity of HBV and had highly toxic effects on kidney, spleen and lung and less toxicity on the liver.

As used herein, the term “fractionation” means a separation process in which a certain mixture is divided into a number of smaller quantities (fractions). The term “protein purification” as used herein refers to a technique by which a single protein type is isolated from a complex mixture. Therefore, the protein fraction may contain two (For example, PF₃₀ and PF₅₀) or more (For example, PF₆₀ and the CPF) proteins. The purified protein or fraction means single protein with fewer impurities (For example MRJP2 and its isoform X1).

The term “iso form” as used herein means a protein that has the same function as the original protein but which is encoded by a different gene and may have small differences in its sequence. Here the MRJP2 and its isoform X1 are encoded by different genes and having slightly different in their sequences.

The term “predicted protein” means the protein which its functional expression is not yet shown in experimental studies and its prediction occurred by automated computational analysis for the genome of the organism.

The term “hepatocytes” refers to the epithelial parenchymatous cells of the liver which make up 70-85% of the liver's mass and responsible for most of the liver functions.

The term “collagen” refers to the main structural protein in the extracellular space in the various connective tissues in animal bodies. As the main component of connective tissue, it is the most abundant protein in mammals. Glycine, proline, hydroxyproline, and hydroxylysine are the main amino acid composition of collagen.

The term “dialysis” means the separation of substances in solution by means of their unequal diffusion through semipermeable membranes. For example, a separation of colloids from soluble substances. Here, the prepared protein fractions from RJ were dialyzed using a semipermeable membrane to remove the impurities from the isolated proteins such as ammonium sulfate, protease inhibitors, and all other associated small molecules.

The term “lyophilized” or “freeze dried” means to dry something (For example, food) in a frozen state under high vacuum especially for preservation. In this invention, the isolated fractions from RJ were freeze dried to obtain the powdered form for accurate preparation of different concentrations for the analyses.

As used herein, the term “crude protein” means all the water soluble proteins in the RJ.

The term “modified protein” refers to the proteins which subjected to the post-translational modification process through the attachment to specific non-protein group. Glycoproteins are that contain oligosaccharide chains (glycans or sugars) covalently attached to polypeptide side-chains. The process of attachment of sugars to the protein named glycosylation.

The term “proteolysis” or “proteolytic” refers to the hydrolysis of proteins or peptides with the formation of simpler and soluble products (digest). This process can be occurred enzymatically using trypsin, a serine protease that specifically cleaves proteins (enzyme substrate) at the carboxylic side of lysine and arginine residues. Here, the sequencing modified grade trypsin was used which is a modified trypsin by reductive methylation of the lysine residues in the porcine native enzyme yielding a highly active and stable molecule. The resulting enzyme is extremely resistant to autolytic digestion to provide maximum specificity for the PMF analysis by MALDI-TOF.

“PI” or “isoelectric point” of a protein means the pH at which the positive charge of the protein balances with its negative charge.

### DETAILED DESCRIPTION

This invention provides two purified proteins from RJ (obtained from the local market, Egypt) having high potency in the prevention of HCV and HBV replication and improving their related liver diseases, fibrosis and cancer.

### RJ Fractionation

RJ was separated into three distinct fractions, sugars, lipids and proteins and their yields are recorded in Table 1.

For the preparation of sugar fraction, 2 g of RJ was dissolved in water/methanol mixture (3:1) and deproteinized using Carrez I (potassium hexacyanoferrate II) and Carrez II (zinc acetate) reagents. Then, lipids were removed by washing the deproteinized RJ two times with dichloromethane. The aqueous layer (sugar fraction) was filtered through 0.2 μm disposable syringe filter, quantified, lyophilized (Telstar, Terrassa, Spain) and kept at −80° C. until used.

Lipids were isolated from RJ with petroleum ether using Soxhlet apparatus for 30 mM. The organic solvent was evaporated, and then the lipid fraction was weighed and stored at −80° C.

The water soluble proteins were extracted from RJ using ammonium sulfate crystals (Brixworth, Northants, UK). In brief, 1.5 g of RJ was dissolved in phosphate buffer saline (PBS, 0.1 M, pH 7) containing 1× protease inhibitor cocktail (Sigma-Aldrich, St. Louis, Mo., USA) and the solution was centrifuged at 3800 g and 4° C. for 30 min. Then the water soluble proteins in the supernatant were precipitated by adding crystals of ammonium sulfate until the saturation reach to 60%. Pellet (crude protein fraction, CPF) was dissolved in PBS, dialyzed for 24 h against the same buffer and finally freeze dried to obtain the powdered fraction. The protein content in the prepared fraction was quantified using Bradford's method.

The three prepared fractions (sugars, lipids, and proteins) were tested for the anti-HCV efficacy and the results have proven the potency of the CPF only.

### Fractionation of RJ CPF

To know the effective anti-HCV protein in the CPF, it was subjected to ammonium sulfate precipitation using different degrees of saturation (5, 10, 15, 20, 30, 50 and 60%). The precipitated proteins were obtained by centrifugation at 3800 g (4° C.) for 30 min and the protein content was examined in each fraction using Bradford method. The fraction containing detectable protein content (30, 50 and 60) were dialyzed for 24 h against PBS, lyophilized and then analyzed with sodium dodecyl sulfate-polyacrylamide gel electrophoresis (SDS-PAGE). FIG. 2 revealed that the PF, which obtained using 30% saturation of ammonium sulfate (PF₃₀) separated as two protein bands and the same two bands with higher concentration present in PF₅₀. However, these two bands in PF₃₀ and PF₅₀ are absent in PF₆₀ that separated as different eleven protein bands. The three PFs were studied for their activities against HCV and the results showed the potency of PF₃₀ and PF₅₀ only while PF₆₀ showed negative anti-HCV effect. These two PFs as mentioned before had the same two protein bands and differ in their concentrations. Therefore, to identify the responsible anti-HCV protein, the higher protein content fraction (PF₅₀) was further fractionated into two purified proteins to evaluate their efficacy against HCV.

### Purification of MRJP2 and its Isoform (MRJP2 X1)

The purification method of MRJP2 and its isoform from RJ is a novel method.

PF₅₀ was used for purification of MRJP2 and MRJP2 isoform X1 using carboxymethyl (CM)-Sephadex ion-exchange column chromatography. The amount of PF₅₀ that obtained from 10 g of RJ was dissolved in 20 mL of the binding buffer (20 mM phosphate buffer containing 1× protease inhibitor cocktail, pH 6.7). The protein solution then applied to CM-Sephadex column (16×2.5 cm) and left for 1 h at 4° C. The unbound protein (MRJP2 isoform X1, fraction 1) was obtained by washing the column with about 100 mL of the binding buffer. Elution of the bound protein (MRJP2, fraction 2) was achieved by a one-step gradient of about 50 mL of 0.5 M NaCl in the binding buffer. The protein content was determined in the purified fractions by UV measurement at 280 nm after dialysis for 24 h against PBS (pH 7). Both dialyzed fractions were freeze dried, then quantified by Bradford and analyzed by SDS-PAGE. The results showed that the protein content in 1 mg of the fraction 1 and fraction 2 powders were 0.95 and 0.5 mg protein, respectively. This observation indicates that nearly half the weight of fraction 2 (MRJP2) is non-protein which means that MRJP2 is a modified protein. Several studies reported the glycosylation of MRJP2 at several sites, therefore; the loss in weight of fraction 2 powder refers to the attached carbohydrates. In addition, MRJP2 isoform X1 may be unmodified or slightly modified protein.

### Identification of MRJP2 and its Isoform (MRJP2 X1)

These two proteins were identified through determination of their molecular weights by SDS-PAGE and matrix-assisted laser desorption/ionization time of flight mass spectrometry (MALDI-TOF MS). In addition, peptide mass fingerprinting (PMF) after tryptic digestion was used for identification of these proteins using MALDI-TOF MS instrument.

### SDS-PAGE

The prepared CPF and each of its precipitated PFs were analyzed by 14% SDS-PAGE (FIG. 2). For each sample, 30 μg of protein were loaded and electrophoresed at 75 V through stacking gel followed by 125 V for about 2 h. The gel was stained with Coomassie brilliant blue R-250 and the molecular weights of the prepared PFs were analyzed with gel documentation system (Geldoc-it, UVP, England) using Totallab analysis software (version 10.1). The results showed that the molecular mass of MRJP2 is 49.949 kDa and its isoform has the mass of 53.117 kDa.

Matrix assisted laser desorption ionization-time of flight mass spectrometry

### MALDI-TOF Ms

The MALDI-TOF Ms was used to identify the purified proteins from PF₅₀ through their molecular weights and their PMF after tryptic digestion. In-solution digestion of the proteins was performed by dissolving 200 μg of each lyophilized PF in 100 μL of 50 mM sodium bicarbonate (pH 8.0). Cysteines were reduced by 5 μL of DTT (45 mM) and alkylated by 5 μL of iodoacetamide (IAA, 100 mM), and then another amount of DTT was added to destroy the excess of IAA. Afterward, proteins were subjected to proteolysis by trypsin modified sequencing grade at a (enzyme:substrate) ratio of (1:50) and incubated at 37° C. for 24 h. The reaction was terminated by addition of 10 μL of 10% trifluoroacetic acid (TFA). One microliter of the tryptic digest was added to the preloaded MALDI plate with 14, of the MALDI matrix (saturated solution of α-cyano-4-hydroxycinnamic acid in 2.5% TFA and 50% acetonitrile) and air dried. The masses of the tryptic peptides were determined using MALDI-TOF MS UltraFlex system (Bruker Daltonics GmbH, Bremen, Germany). The analysis was done in the linear positive ion mode in the mass/charge (m/z) range of 500-4000 Da using FlexControl software version 3. The generated spectra were compared to the database (fingerprint) using the Bruker Biotyper software (version 3.1) and a library of 5,623 entries.

The results obtained in FIG. 3, 4 demonstrated the mass and the matched tryptic peptides with the sequence of MRJP2 and its isoform X1. Analysis showed five matched peptides from nineteen tryptic peptides (11.5% sequence coverage) for MRJP2 and eight matched peptides from thirty-five tryptic digest (20.8% sequence coverage) for MRJP2 isoform X1. The calculated molecular masses of both proteins from the tryptic peptides masses are 32.716 kDa for MRJP2 and 57.115 kDa for MRJP2 X1. From these results, we can notice the mismatch between the masses obtained by MALDI-TOF and SDS-PAGE analyses. The slight differences in the mass value of MRJP2 isoform X1 may be owed to the difference in the protein shape in both analyses due to the degradation of the protein disulfide bonds by SDS. In addition, these two proteins or one of them is glycoprotein attached to carbohydrate moieties when bind to SDS gives SDS-micelle which changes the protein shape. It is well-known that the shape of the protein is an important factor for accurate mass determination. However, the large difference in mass values of MRJP2 by both analyses probably related to the presence of large oligosaccharides attached to this protein (confirmed by previous studies) and shields the trypsin proteolytic cleavage sites. This shielding effect leads to a lower number of peptides and yields poor peptide mass maps by MALDI-TOF analysis. No doubt that the MALDI-TOF analysis for protein mass is more accurate than SDS-PAGE, but false results may be obtained for glycoproteins. On the other hand, the isoelectric point (PI) values were obtained for MRJP2 (PI 7) and its isoform (PI 6.5) from the MALDI-TOF MS analysis.

### Anti-HCV and Anti-HBV Activities

The present study evaluated the anti-HCV and anti-HBV effects of RJ and its isolated fractions for identification of the responsible ingredients. The anti-HCV activity of RJ and its isolated fractions include lipids, carbohydrates, CPF, PF₃₀, PF₅₀, PF₆₀, MRJP2 and MRJP2 isoform X1 in comparison with SOV was examined qualitatively and quantitatively. Then, the most effective anti-HCV RJ fractions were studied for their anti-HBV effects quantitatively. The antiviral activity was evaluated in vitro using two types of host cells, PBMCs and HepG-2.

### Viral Host Cells Isolation and Culture

PBMCs were obtained by Ficoll-Hypaque density gradient centrifugation method as described previously. In brief, the blood samples from healthy volunteers were diluted with equal volume of PBS, carefully layered on Ficoll-Hypaque, and centrifuged at 2000 rpm, 25° C. for 30 min. Then the undisturbed PBMCs layer (interface) was carefully transferred out, washed with 40 ml RPMI-1640 medium, and centrifuged at 1650 rpm for 10 min. Finally, the supernatant was removed and the cells were suspended in 5 ml of RPMI-1640 medium containing 10% FBS and counted using trypan blue stain.

HepG-2 cells were grown in RPMI-1640 medium (HyClone) supplemented with 10% heat-inactivated FBS.

### Infectious Serum Samples

Serum samples were obtained from HCV and HBV infected Egyptian patients “A.R.” after agreement by the ethics committee. The HCV and HBV samples were positive for genotype 4a and D, respectively. The infected patients were neither under treatment prior to the study nor co-infected. The sera were kept at −80° C. until use.

### In Vitro Infection with HCV and HBV

The viral host cells, human PBMCs (1×10⁶ cells) and HepG-2 (1.5×10⁵ cells) were seeded in each well of 12-well culture plate and 6-well culture plate, respectively. All wells were incubated in the CO₂ incubator (New Brunswick Scientific, Netherlands) with either HCV (2.9×10⁵ copies/ml) or HBV (1×10⁵ copies/ml) infected serum in RPMI-1640 medium for 2 h at 37° C., 5% CO₂ and 95% humidity.

### Qualitative Screening for HCV

After cellular infection, the infected medium was replaced with a fresh RPMI-1640 medium containing 10% FBS for positive control wells. For treated wells, the infected medium was exchanged with RPMI-1640 medium containing 10% FBS and 200 μL of RJ (0.2 and 1 mg), or one of its fractions including carbohydrates, lipid and CPF (at a dose equivalent to their amounts in 0.2 mg RJ). Additionally, 1 mg of PF_{30, 50, 60}, 250 μg of MRJP2 and MRJP2 X1 or 4 mg of SOV were tested. Two control cultures were included, positive (infected cells only) and negative (uninfected cells). All cells were incubated at 37° C., 5% CO₂ and 95% humidity for 72 h, then the total RNA was extracted from the treated and untreated cells following the Gene JET RNA purification Kit protocol (Thermo Scientific, USA). The HCV (+) strand was detected by reverse transcription-nested polymerase chain reaction (RT-nested PCR) using the Ready-To-Go RT-PCR beads (Amersham Pharmacia Biotech, Piscataway, N.J., USA) following the instructions. The primer sequences that used in this PCR are derived from the highly conserved 5′-untranslated region (5′-UTR) of HCV genome. Finally, the PCR products were electrophoresed on 2% agarose gel and the gel image was visualized on a UV transilluminator and photographed using gel documentation system.

Results revealed the anti-HCV effect of RJ at the high and low concentrations, CPF, PF₃₀, PF₅₀, MRJP2, MRJP2 isoform X1 and SOV and the other studied fractions had no anti-HCV activity. The positive HCV samples revealed one band with a molecular size of 174 bp [(+) strand RNA amplified products] (FIG. 5).

### Quantitative Analysis for the Intracellular HCV and HBV

Different concentrations of RJ (0.2 and 1 mg), PF₅₀ (0.25, 0.5, 1 mg), MRJP2 and its isoform X1 (250 μg, 100, 50, 25 ng) were incubated with the HCV-infected PBMCs or HepG-2 cells. In addition, PF₅₀ (1 mg), MRJP2 and MRJP2 isoform X1 (250, 500 μg) were incubated with HBV-infected cells. SOV at the concentration of 4 mg was incubated only with HCV or HBV-infected PBMCs due to its potent anti-cancer effect. All cells were incubated for 72 h in the CO₂ incubator. Then the HCV-RNA and HBV-DNA within these cells were quantified using the fully automated Cobas Ampliprep TaqMan Analyzer (CAP-CTM). This technique is one of the most recent techniques that used for diagnosis of viral hepatitis quantitatively. All steps were automated and include the extraction of RNA or DNA on the Cobas AmpliPrep instrument and simultaneously, the PCR amplification and detection on the Cobas TaqMan analyzer. The procedure was done following the manufacturer's instructions.

To examine the effect of RJ and its protein fractions on the viral entry, 1 mg of each of PF₅₀, MRJP2 and MRJP2 isoform X1 or 4 mg of SOV were incubated with PBMCs or HepG-2 (not used with SOV) for 10 min before infection. These cells were infected as described above with either HCV or HBV in RPMI-1640 medium for 2 h in the CO₂ incubator. Then the medium was discarded and medium containing 10% FBS was added to all wells. Two control cultures were included and incubated at the same conditions, positive (infected cells only) and negative (uninfected cells). After 24 h incubation, cells were washed and the viral load within the cells was quantified by fully automated Cobas Ampliprep TaqMan Analyzer (CAP-CTM). Tables (2-5) summarize the obtained results.

### In Vitro Assessment of Liver Fibrosis

The anti-fibrotic effect of RJ and its protein fractions (PF₅₀, MRJP2, and MRJP2 isoform X1) and SOV in comparison with silymarin (SM) standard drug were studied using hepatocytes.

### Isolation of Hepatocytes and Cytotoxicity Assay

Hepatocytes were isolated from the liver of two male Albino rats 1 week of age. Rats were anesthetized, then liver was dissected, washed and incubated for 15 min with 10× penicillin and streptomycin. Afterward, the liver was washed using PBS, minced and incubated for 30 min with 3 U/mL of collagenase I with gentle shaking to obtain the cell suspension. The solution was centrifuged for 5 mM at 1000 rpm and the pellet (hepatocytes) was washed, suspended in William's E medium containing 10% FBS and incubated at 3TC in CO₂ incubator. After 90% cell confluent, hepatocytes were passaged with trypsin-EDTA. Then cells were stained with trypan blue, checked for viability and counted using the phase contrast inverted microscope (Olympus IX 81, Tokyo, Japan).

The cytotoxicity of the studied compounds on the hepatocytes was done using MTT (3-(4,5-dimethylthiazol-2-yl)-2,5-diphenyltetrazolium bromide) assay. In brief, about 10⁴ cells/well were seeded in 96-well cell culture plate and treated separately with serial dilutions of RJ, PF₅₀, MRJP2, MRJP2 X1, SOV, and SM. In addition, the untreated cells were included as negative control. All plates were incubated in the CO₂ incubator at 5% CO₂, 37° C., and 90% relative humidity and after 72 h, 20 μL of 5 mg/mL MTT was added/well and incubated for further 4 h. Then plates were centrifuged at 2000 rpm for 10 min and 150 μl DMSO was added to each well after supernatant aspiration and the absorbance was read at 570 nm using ELISA reader (BMG LabTech, Germany). Cell viability was determined and the safe concentrations (EC100, 100% cell viability) were calculated (Table 6).

### Induction of Fibrosis and Treatment Protocol

For induction of fibrosis, hepatocytes were incubated with various concentrations (0.13-1.30 mM) of CCl₄ for 48 h. At the end of the incubation period, the cell viability was assessed and the IC₅₀ value (half maximal inhibitory concentration of CCl₄ to the hepatocytes growth) was calculated by the GraphPad Instat software version 3. This value was used for in vitro induction of fibrosis in hepatocytes (FM). After induction, Fb1 was incubated with the safe concentration of each of the studied compounds and incubated for 72 h at 5% CO₂ and 37° C. The normal cells and the untreated Fb1 (Fb) were involved as negative and positive control cells, respectively.

### Biochemical Analyses and Identification of Apoptosis

At the end of the incubation period, media were collected and used in the assessment of the hepatocyte damage indices. However, cells were examined morphologically for identification of apoptosis.

Alanine and aspartate aminotransferases (ALT and AST), and albumin were determined using available commercial kits (RAM, Egypt). TNF-α (ELISA kit, RayBiotech, USA) and collagen IV (ELISA kit, Kamiya Biomedical) were determined following the manufacturer's instructions. Nitric oxide (NO) level was assessed by measurement of the nitrite using the Griess reaction, which produced colored azo dye with a maximum absorbance at 490 nm. Hydroxyproline content in the medium was measured using chloramines T and dimethylaminoborane solutions. The absorbance of the produced color was read spectrophotometrically at 560 nm and the concentration was determined from the hydroxyproline calibration curve.

Apoptosis was investigated by double staining cells after washing with PBS with 100 μg/mL of ethidium bromide (EB) and acridine orange (AO) as described previously and then visualized under the fluorescent phase contrast microscope.

As shown in FIG. 6-9), the treatment with MRJP2 or its isoform resulted in the following:
1. Improvement in the hepatocytes morphology, which indicated by the decrease in the number of the early and late apoptotic cells as compared with the induced cells (Fb).
2. Improvement in the liver function parameters.
3. Normalization of the inflammatory mediators' level.
4. The therapeutic effects of the RJ proteins were similar to or slightly less than the standard drug (SM), but significantly more effective than SOV.

### Anti-Cancer Assay

The cytotoxic effect of RJ and its isolated PFs against HepG-2 cell line were evaluated. The cell viability was determined by MTT assay to investigate the cytotoxicity. Cells (3×10³/well) were seeded in 96-well plate and left to attach for overnight. Serial concentrations (6.25-100 μM) of each of the tested compounds were added to the attached cells and incubated for 72 h at 37° C. in an atmosphere of 5% CO₂ and the untreated cells were included as negative control. Then, 20 μl of MTT solution (0.5 mg/ml) was added to each well and the plate was incubated at 37° C. and 5% CO₂ for 4 h. The medium with MTT was replaced by 150 μl of DMSO and the absorbance of the produced color was read at 570 nm by the microplate reader. The concentration of each of the studied compounds that inhibit the cancer cell growth by 50% (IC₅₀ values) was determined from the dose-response curves. Additionally, the morphological changes of the tumor cells were examined using the phase-contrast microscope.

The results in FIG. 10 and the IC₅₀ values in Table 6 showed the cytotoxic effects of both MRJP2 and its isoform X1. The results showed that MRJP2 was nearly as potent as SOY and 5-FU, but its isoform was significantly less potent than them.

### Assessment of Acute Toxicity of RJ and SOV

Karber's method was used to study the acute toxicity effect of RJ in comparison with SOV using male Albino rats and the median lethal dosage (LD50) was calculated.

### Experimental Animals and Design

Fifty-five male Albino rats (80-120 g) were purchased from MISR University for Science and Technology (animal welfare assurance number A5865-01). Animals were housed in metal cages and allowed free access to a standard commercial diet and tap water. Rats were kept under conventional conditions of temperature, humidity and twelve hours light/dark cycle. All rats were acclimatized to the laboratory environment for one week prior to handling and were observed daily for abnormal signs. Handling of animals complied with the international guide for the care and use of laboratory animals (National Research Council, 1996).

Animals were divided into eleven groups (five animals in each) starting from group I to group X beside the control (received only water as the vehicle). Groups from (I) to (V) were administered RJ intraperitoneally, while groups from (VI) to (X) were orally received SOV using the gavage tube. RJ and SOV were given to the animals at various doses (140, 350, 700, 2500 and 5000 mg/kg bw), one dose for each group of animals (group I and VI received the lowest dose).

### Assessment of Hematological and Biochemical Parameters

All animals were left for 24 h after RJ or SOV administration, and then the number of mortality in each group was recorded for calculation of the LD₅₀ value. Also, the weights of animals in each group were recorded before sacrificing by decapitation. Blood samples were collected in heparin tubes by cardiac puncture for assessment of the erythrocytes, hematocrit (HCT), hematimetric indices, hemoglobin (Hb), platelets, and leukocytes (full and differential counts). In addition, ALT and AST, total proteins, albumin, urea, creatinine, sodium, potassium, triglycerides (TG) and cholesterol were determined using available commercial kits (RAM, Egypt). The vital organs including liver, lung, kidney, and spleen were carefully excised, weighed and examined.

The following results were obtained:
1. There was no mortality occurred after 24 h from receiving either RJ or SOV at all the studied doses. The LD50 value of both RJ and SOV is >5000 mg/kg bw.
2. Administration of RJ and SOV at all doses didn't cause any significant change in the body weights of the animals as compared with the control group (FIG. 11).
3. Administration of RJ is safe on the rats' vital organs, but SOV is highly toxic (FIG. 12).
4. Slight changes in the hematological parameters after either administration (Table 7, 8).
5. The liver function parameters didn't change after 24 h from RJ administration with all doses (Table 7). Nevertheless, the doses of 2500 and 5000 mg/kg bw of SOV significantly reduce the serum level of albumin and significantly elevated the activity of AST more than the normal control and didn't affect ALT activity and total proteins level (Table 8).
6. RJ didn't induce any significant abnormal kidney function parameters. Interestingly, its high dose (5000 mg/kg bw) significantly decreased the serum urea level and all given doses significantly decreased the sodium level in the blood (Table 7). Therefore, RJ enhances the kidney function of the treated animals. In contrast, SOV induced a significant elevation in the serum urea level when given in doses of (350-5000 mg/kg bw) and the higher doses (2500, 5000 mg/kg bw) also elevated the serum sodium level and depleted potassium level (Table 8).
7. The administration of RJ at the doses of (140-5000 mg/kg bw) significantly decreased the TG level in the blood but didn't affect the cholesterol level (Table 7). However, SOV administration nearly at all doses significantly increased the levels of TG and cholesterol (Table 8).

In summary, RJ has been shown to be safe at a dose higher than 5 g/kg in rats. However, SOV is toxic at all the studied doses starting from the dose of 0.14 g/kg of rat bw and the most affecting organs by it are kidney, spleen, and lung while the liver is the less affecting one.

### Statistics

Data were expressed as mean±SE and were analyzed by SPSS version 16. The mean values were compared using one-way analysis of variance (ANOVA) by Duncan's test and significance was determined at P<0.05. IC₅₀ and EC₁₀₀ values were calculated by the GraphPad Instat software version 3.

### BRIEF DESCRIPTION OF THE DRAWING

FIG. 1: Apis mellifera royal jelly.

FIG. 2: SDS Polyacrylamide gel electrophoresis (SDS-PAGE) of royal jelly protein fractions. Lane 1, molecular mass standards; Lane 2, crude protein fraction (CPF, precipitated from RJ using 60% ammonium sulfate); Lane 3, 4, 5, protein fraction 30, 50, and 60 (obtained by precipitation of CPF with 25-30%, 40-50% and 50-60% of ammonium sulfate), respectively; Lane 6, 7, major royal jelly protein (MRJP) 2 isoform X1 and MRJP2, respectively (purified from RJ protein fraction 50 by ion exchange chromatography).

FIG. 3: MALDI-TOF MS spectrum of the major royal jelly protein (MRJP) 2. (A) Showed the amino acid sequence of MRJP2 indicates the five matched peptides of the spectrum and their coverage was 11.5%. (B) Showed the mass spectra of the peptides obtained from trypsin digestion of MRJP2. (C) The Table showed the masses of the MRJP2 tryptic digests and their intensities.

FIG. 4: MALDI-TOF MS spectrum of the major royal jelly protein (MRJP) 2 isoform X1. (A) Showed the amino acid sequence of MRJP2 isoform X1 indicates the eight matched peptides of the spectrum and their coverage was 20.8%. (B) Showed the mass spectra of the peptides obtained from trypsin digestion of MRJP2 isoform X1. (C) The Table showed the masses of the MRJP2 isoform X1 tryptic digests and their intensities.

FIG. 5: Agarose gel electrophoresis images of RT-nested PCR for detection of HCV (+) strand RNA using (A) Peripheral blood mononuclear cells, PBMCs and (B) HepG-2 cells. For both cells, Lane M, is the molecular weight marker (M) 100 bp ladder; Lane 1, is a negative control; lane 2, is a positive control; Lane 3,4 shows the effect of royal jelly at 1 mg and 0.2 mg, respectively on the HCV-RNA (+) strand; Lane 5-7, shows the effect of royal jelly lipid, carbohydrate and crude protein fraction (CPF, precipitated from RJ using 60% ammonium sulfate), Lane 8-10, PF₃₀, PF₅₀, and PF₆₀, respectively (obtained by precipitation of CPF with 25-30%, 40-50% and 50-60% of ammonium sulfate, respectively). Lane 11 (A), shows the effect of Sovaldi at 4 mg; Lane 12 (A), 11 (B), shows the effect of major royal jelly protein 2 at 250 μg. Lane 13 (A), 12 (B), shows the effect of major royal jelly protein 2 isoform X1 at 250 μg. We cannot study the effect of SOV on HepG-2 cells due to its potent anticancer effect. This FIG. revealed the anti-HCV effect of RJ, CPF, PF₃₀, PF₅₀, MRJP2 and its isoform X1 and SOV at the used concentrations.

FIG. 6: Detection of apoptosis in the hepatocytes by acridine orange/ethidium bromide double staining using fluorescence microscope (magnification 200×). (1), Untreated viable cells are uniformly pale-green; (2), Early apoptotic cells stained bright-green to yellow and have characteristic chromatin condensation and loss of membrane integrity; (3), Late apoptotic cells stained yellow-orange or red color, with a fragmented or condensed chromatin; (4), Necrotic cells stained bright orange-red. Control, untreated cells; Fb1, fibrotic cells (hepatocytes after 48 h from CCl₄ treatment); Fb, fibrotic cells (hepatocytes after 120 h from CCl₄ treatment); Fb1-RJ, Fb1 after treatment with royal jelly (200 μg/mL); Fb1-PF₅₀, Fb1 after treatment with 200 μg/mL of protein fraction 50 (obtained by precipitation of crude RJ proteins with 40-50% ammonium sulfate); Fb1-MRJP2, Fb1 after treatment with major royal jelly protein 2 (200 μg/mL); Fb1-MRJP2 (X1), Fb1 after treatment with major royal jelly protein 2 isoform X1 (200 μg/mL); Fb1-SOV, Fb1 after treatment with Sovaldi (200 μg/mL); Fb1-SM, Fb1 after treatment with Silymarin (60 μg/mL). The results showed the enhancing effect of the PF₅₀ and its PFs (MRJP2 and its isoform X1) to the CCl₄-induced apoptosis in hepatocytes and the less potency of SOV.

FIG. 7: Effect of royal jelly (RJ) and its protein fractions, Sovaldi (SOV) and Silymarin (SM) on collagen and hydroxyproline levels in CCl₄-induced fibrosis in hepatocytes (Fb1). PF₅₀, RJ protein fraction 50 (obtained by precipitation of crude RJ proteins with 40-50% ammonium sulfate); MRJP2, major royal jelly protein 2; MRJP2 (X1), major royal jelly protein 2 isoform X1. Data are expressed as mean±SE (n=3). Different letters are significantly different for the same parameter at P<0.05. The FIG. showed the suppressive effect of RJ and its PFs and SOV for collagen and hydroxyproline levels in the fibrotic hepatocytes.

FIG. 8: Changes in some inflammatory mediators in fibrotic cells (Fb1, hepatocytes after 48 h from CCl₄ treatment) after the treatment with royal jelly (RJ) and its protein fractions, Sovaldi (SOV) and Silymarin (SM). PF₅₀, RJ protein fraction 50 (obtained by precipitation of crude RJ proteins with 40-50% ammonium sulfate); MRJP2, major royal jelly protein 2; MRJP2 (X1), major royal jelly protein 2 isoform X1; NO, nitric oxide; TNF-α, tumor necrosis-α. Data are expressed as mean±SE (n=3). Different letters are significantly different for the same parameter at P<0.05. Results showed the anti-inflammatory effect of RJ and its PFs and SOV by reducing NO and TNF-α levels.

FIG. 9: Liver function parameters in fibrotic cells (Fb1, hepatocytes after 48 h from CCl₄ treatment) after the treatment with royal jelly (RJ) and its protein fractions, Sovaldi (SOV) and Silymarin (SM). PF₅₀, RJ protein fraction 50 (obtained by precipitation of crude RJ proteins with 40-50% ammonium sulfate); MRJP2, major royal jelly protein 2; MRJP2 (X1), major royal jelly protein 2 isoform X1. Data are expressed as mean+SE (n=3). Different letters are significantly different for the same parameter at P<0.05. The results revealed the ability of RJ PFs but not SOV in improving the liver function parameters. These results mean improving the fibrosis in hepatocytes by these fractions.

FIG. 10: Cytotoxic effect of royal jelly (RJ) and its protein fractions, Sovaldi (SOV) and 5-fluorouracil (5-FU) on HepG-2 cells. PF₅₀, RJ protein fraction 50 (obtained by precipitation of crude RJ proteins with of crude RJ proteins 40-50% ammonium sulfate); MRJP2, major royal jelly protein 2; MRJP2 (X1), major royal jelly protein 2 isoform X1. The results revealed the anticancer activity of RJ and its PFs and the potent activity of SOV. This can be noticed by the ability of these compounds to induce death in the HepG-2 cell line.

FIG. 11: Effect of royal jelly (RJ) and Sovaldi (SOV) administration with different concentrations on the rat body weight gain. Data are expressed as mean±SE (n=5). Different letters are significantly different for the same tested compound at P<0.05. This FIG. showed that there is no significant change in the rat's body weights after 24 h from RJ or SOV administration.

FIG. 12: Effect of administration of (A) royal jelly (intraperitoneal) and (B) Sovaldi (oral) with different concentrations on % organ-body weight ratios. Data are expressed as mean±SE (n=5). Different letters are significantly different for the same organ at P<0.05. The different doses administration of RJ didn't affect the organ/body weight ratio of all the studied organs. While administration of SOV caused significant enlargement of the liver and lung at the higher doses (2500 and 5000 mg/kg bw) and for the kidney with all the given doses (140-5000 mg/kg bw). In addition, starting from the dose of 350 to 5000 mg/kg bw caused enlargement of the spleen.

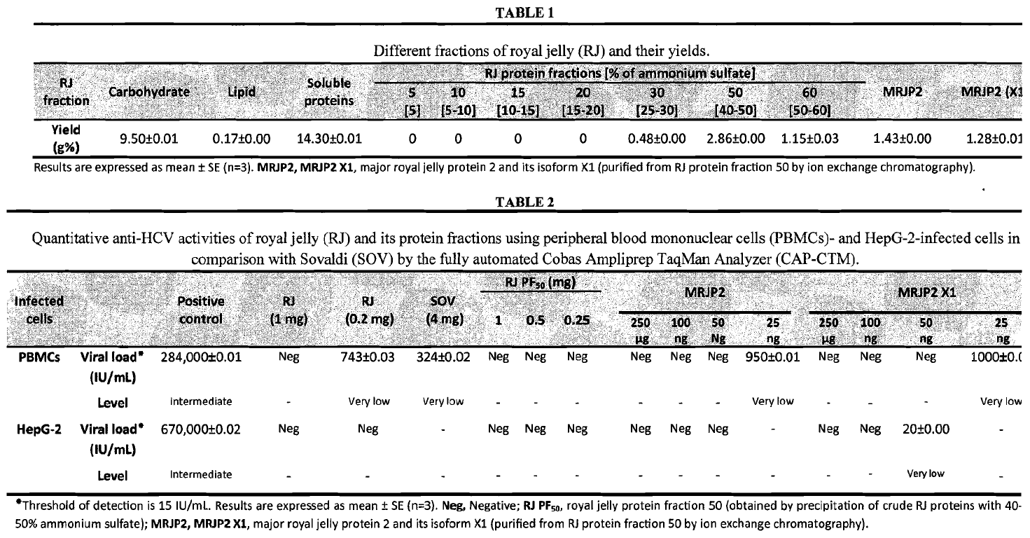

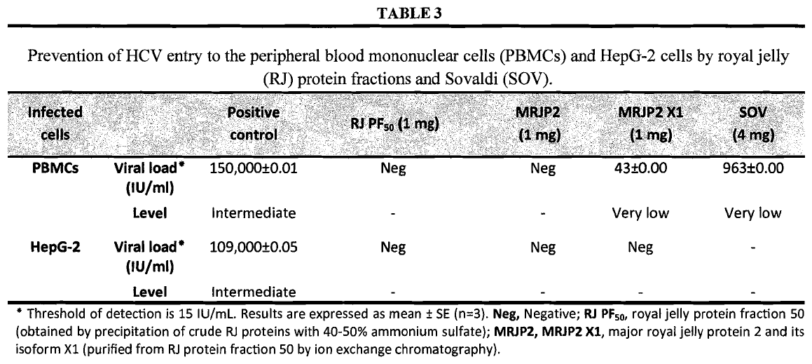

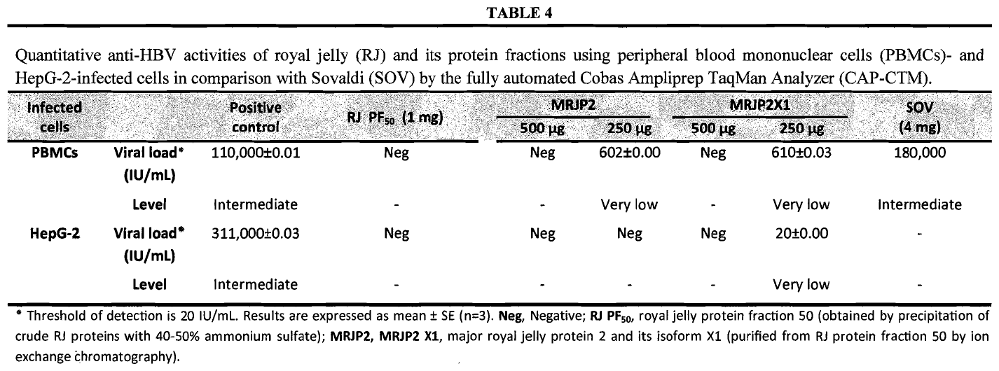

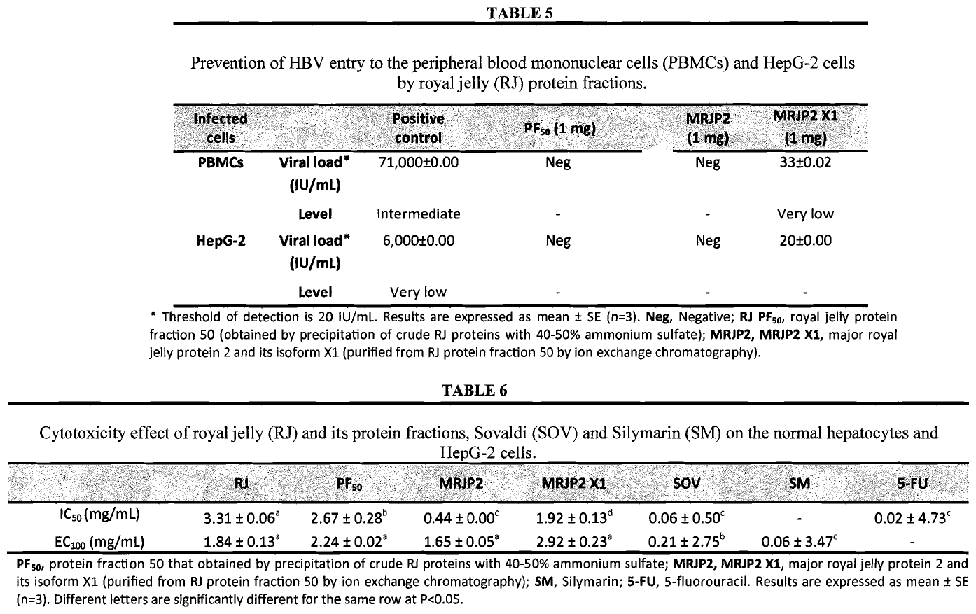

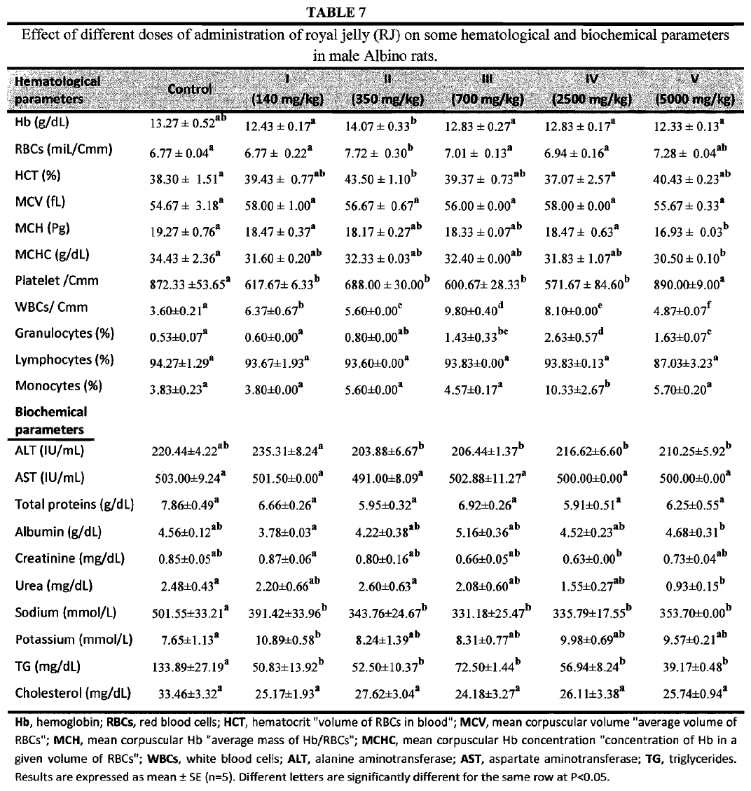

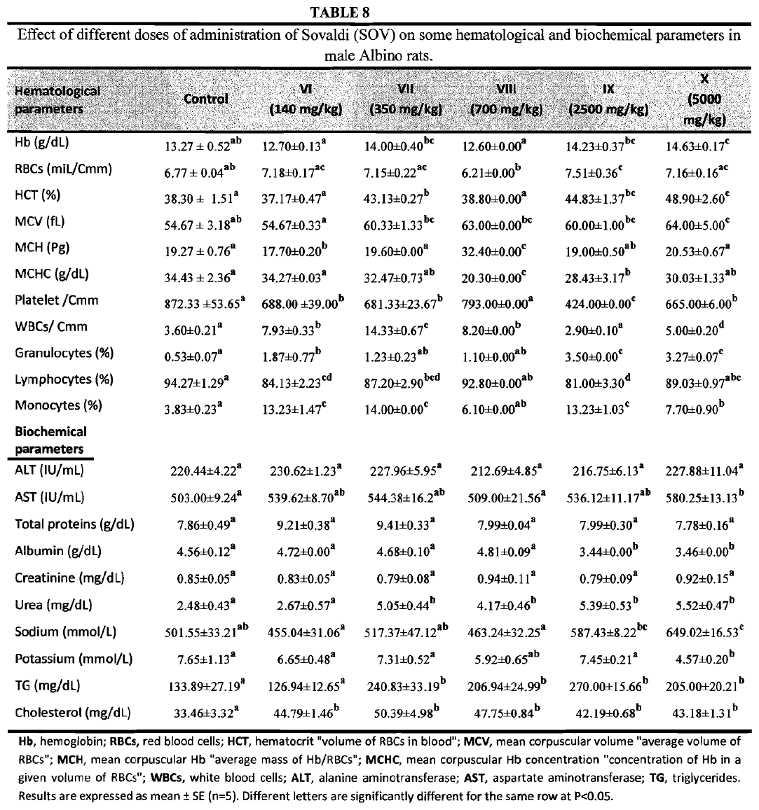

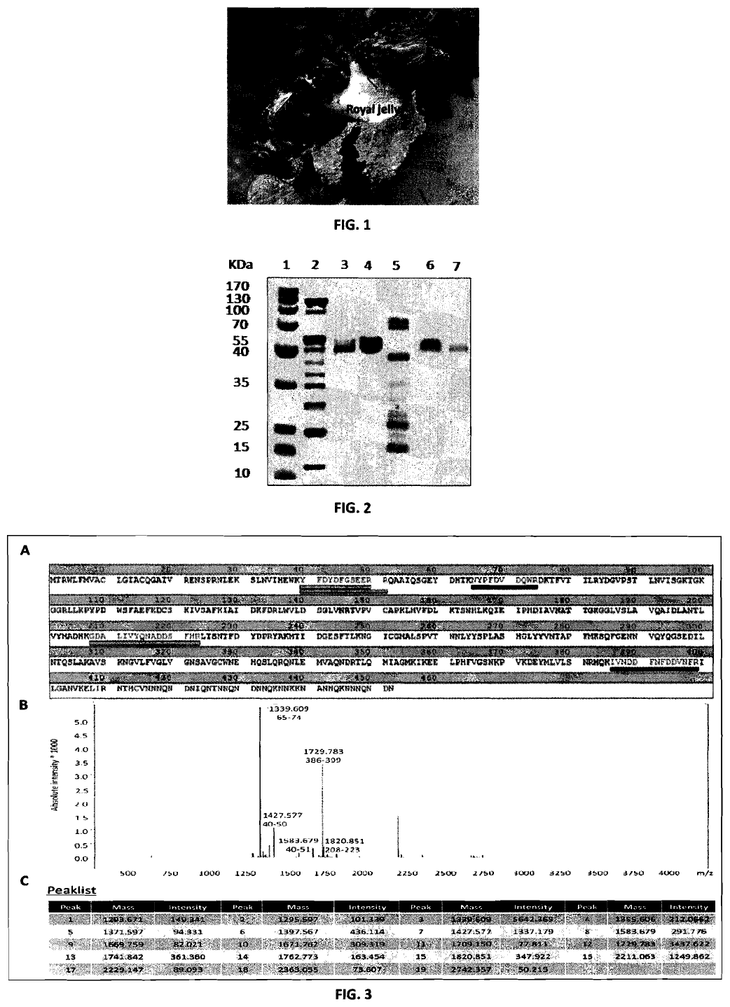

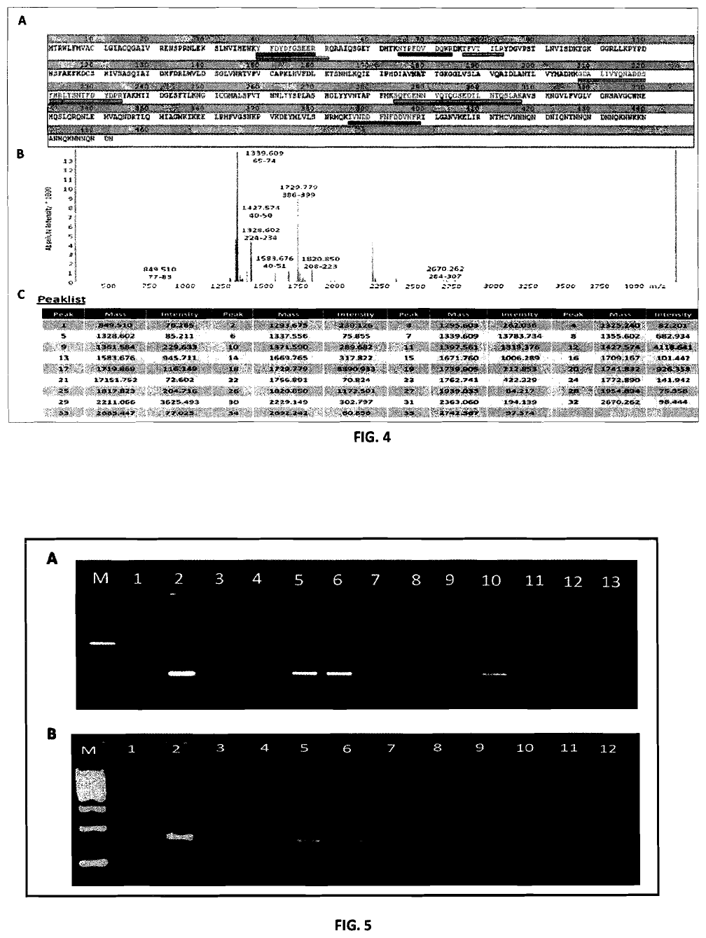

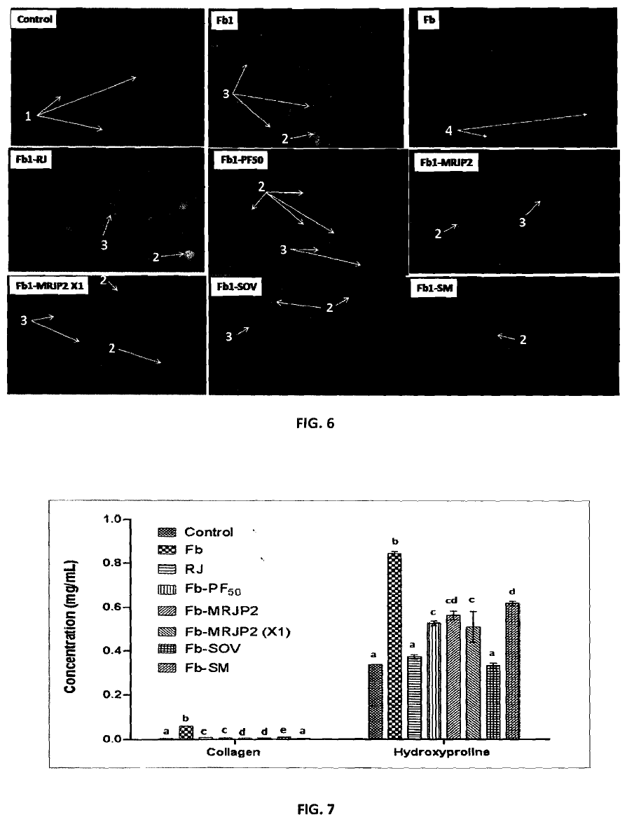

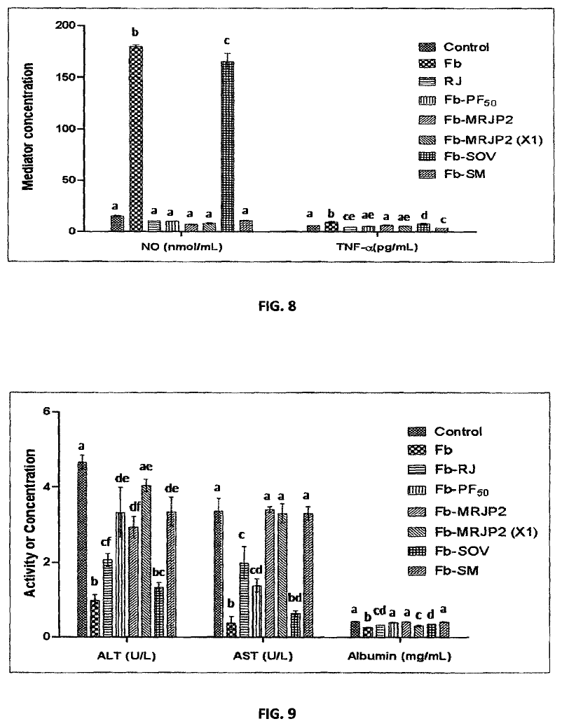

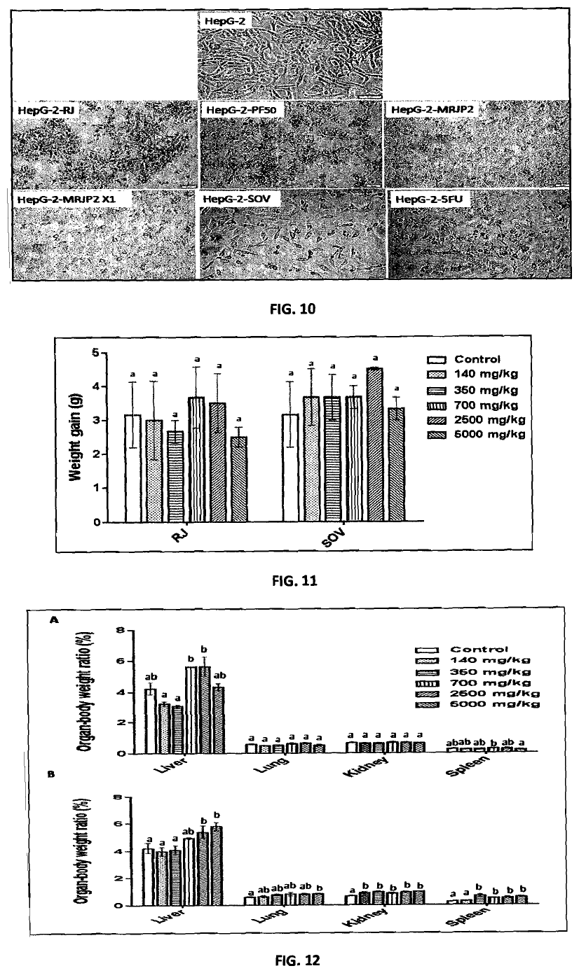

What is claimed is:

1. Two purified proteins from Apis mellifera royal jelly (RJ) named as major royal jelly protein 2 (MRJP2) and its isoform X1 having highly potent preventing effects for HCV, HBV, and their related liver diseases, fibrosis and cancer.

2. A purification method of the effective proteins in claim 1, comprising MRJP2 with its isoform X1 as a single fraction.

3. A purification method of MRJP2 and its isoform X1 as two separate fractions from the fraction obtained in claim 2.

4. A method of inhibiting HCV replication and cellular entry comprising, the use of MRJP2 that purified by the method of claim 3.

5. A method of inhibiting HCV replication and cellular entry comprising, the use of MRJP2 isoform X1 that purified by the method of claim 3.

6. A method of inhibiting HBV replication and cellular entry comprising, the use of MRJP2 that purified by the method of claim 3.

7. A method of inhibiting HBV replication and cellular entry comprising, the use of MRJP2 isoform X1 that purified by the method of claim 3.

8. Anti-HCV and anti-HBV mechanism of the purified proteins of claim 3.

9. A method of improving the liver fibrosis comprising, the use of MRJP2 that purified by the method of claim 3.

10. A method of improving the liver fibrosis comprising, the use of MRJP2 isoform X1 that purified by the method of claim 3.

11. The RJ is safe in the animal model at low and high doses.

12. Sovaldi is a toxic drug for the rat kidney, lung and spleen more than liver.
